(19) United States
(12) Patent Application Publication
(10) Pub. No.: US 2006/0051294 A1
(43) Pub. Date: Mar. 9, 2006

(54) Method of predicting a diet effect

(76) Inventors: Tohru Fushiki, Otsu-shi (JP); Naohiko Inoue, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/218,777

(22) Filed: Sep. 6, 2005

(30) Foreign Application Priority Data
Sep. 6, 2004 (JP) 2004-258935

(51) Int. Cl. A61K 49/00; A61K 36/81

(52) U.S. Cl. 424/9.1; 424/760

(57) ABSTRACT

A method of predicting a diet effect caused by a sympathetic activator includes: making a human ingest the sympathetic activator; obtaining a biological reaction index from the human who has ingested the sympathetic activator; and calculating a predictive value of a diet effect from the biological reaction index based on a regression equation showing a correlation between the preliminarily determined biological reaction index and diet effectiveness.

### BACKGROUND OF THE INVENTION

1. Field of the Invention

The present invention relates to a method of predicting a diet effect, a set of computer-executable instructions (e.g., a ‘program’) for performing the method, and a recording medium in which the program is stored. Specifically, the present invention relates to a method of predicting a diet effect such as an amount of weight loss in a case of ingesting a sympathetic activator such as capsinoid, a program for performing the method, and a recording medium in which the program is stored.

2. Description of the Related Art

It has been reported that a non-pungent fixed variety of capsicum, “CH-19 sweet”, which had been selected and fixed by Yazawa et al. as a capsicum having less pungency, contains few capsaicinoid compounds having a pungency and invasive activity as general capsicums (capsaicin, dihydrocapsaicin, etc.) (hereinafter, often simply referred to as “capsaicinoid”), and instead contains a large amount of novel capsinoid compounds having no pungency (including a fatty acid ester of vanillyl alcohol, capsiate [4-hydroxy-3-methoxybenzyl (E)-8-methyl-6-nonenoate], dihydrocapsiate [4-hydroxy-3-methoxybenzyl 8-methylnonanoate], nordihydrocapsiate [4-hydroxy-3-methoxybenzyl 7-methyl-octanoate], etc., hereinafter, often simply referred to as “capsinoid”) (see JP 11-246478 A). Furthermore, such capsinoid has been confirmed to exist also in other plants belonging to the genus Capsicum (Journal of the Japanese Society for Horticultural Science 58, 601-607).

An experiment with mice has revealed that capsinoid has an enhancing effect on energy metabolism (JP 11-246478 A), weight loss effect (JP 2001-026538 A), increasing effect on oxygen consumption (Biosci. Biotech. Biochem: 65(12) 2735-40 (2001)), etc. Meanwhile, an experiment in which humans ingested “CH-19 sweet” containing capsinoid has confirmed that capsinoid has a hyperthermic effect and increasing effect on oxygen consumption in humans (Biosci. Biotech. Biochem: 65(9) 2033-2036 (2001)). Therefore, capsinoid or a capsicum containing capsinoid has great potential as a diet food.

However, although the hyperthermic effect and increasing effect on oxygen consumption in humans have been confirmed in Biosci. Biotech. Biochem: 65(9) 2033-2036 (2001) above, it is not known whether the degrees of those effects simply represent a body fat reduction effect or weight loss effect in a one-one relation, so that it is impossible to predict a diet effect caused by continuous ingestion of capsinoid from these facts.

Meanwhile, it is generally known that a diet effect varies between individuals, and it takes a long time (several months) before the diet effect is seen. Therefore, a purchaser has difficulty in continuously keeping his motivation to ingest a diet food, and accordingly causes a problem that the effect of the diet food is not seen. If the diet effect can be predicted before ingesting it for a long time, however, motivation to continuously ingest a diet food may be enhanced to fully provide the effect.

### SUMMARY OF THE INVENTION

In view of the aforementioned problems, aspects of the present invention provide a method of predicting a diet effect caused by a sympathetic activator in a human, a program for performing the method, and a recording medium in which the program is stored. More specifically, aspects of the present invention include a method of predicting a diet effect from a biological reaction index obtained from a human who has ingested a sympathetic activator, a program for performing the method, and a recording medium in which the program is stored.

As a result of studies by the inventors of the present invention, it has been found that a biological reaction in a human in a case of ingestion of a sympathetic activator significantly correlates with a diet effect. Among many, some aspects of the present invention include the following.

It is an object of the present invention to provide a method of predicting a diet effect caused by a sympathetic activator including: making a human ingest the sympathetic activator;

obtaining a biological reaction index from the human who has ingested the sympathetic activator; and calculating a predictive value of a diet effect from the biological reaction index, based on a regression equation showing a correlation between a preliminarily determined biological reaction index and diet effectiveness.

It is a further object of the present invention to provide the method as described above, wherein the sympathetic activator is at least one of capsaicinoid and capsinoid.

It is a further object of the present invention to provide the method as described above, wherein the sympathetic activator is selected from the group consisting of a capsicum plant body, a food, a beverage, a pharmaceutical, a cosmetic, a piece of clothing, and a healthcare product, wherein said activator contains at least one of capsaicinoid and capsinoid.

It is a further object of the present invention to provide the method as described above, wherein the sympathetic activator is selected from the group consisting of capsiate, dihydrocapsiate, nordihydrocapsiate and a mixture thereof.

It is a further object of the present invention to provide the method as described above, wherein the biological reaction index is either a sympathetic activity reaction index or a parasympathetic activity reaction index.

It is a further object of the present invention to provide the method as described above, wherein the predictive value of the diet effect is selected from the group consisting of the amount of weight loss, the amount of body fat percentage loss, the amount of body fat amount loss, and the amount of total fat area loss.

It is a further object of the present invention to provide the method as described above, wherein the biological reaction index is a sympathetic activity reaction index, the predictive value of the diet effect is an amount of weight loss, and the regression equation is represented by the following (Equation 1):
Y=aX+b(a is between about 0.0026 and about 0.032, and b is between about −2.9 and about 1.12). (Equation 1)

It is a further object of the present invention to provide a program for predicting a diet effect caused by a sympathetic activator and, when embodied in a set of computer-executable instructions, enabling a computer to perform a method of predicting a diet effect caused by a sympathetic activator, which includes calculating a predictive value of the diet effect from a biological reaction index obtained by a human who has ingested the sympathetic activator based on a regression equation showing a correlation between the preliminarily determined biological reaction index and diet effectiveness.

It is a further object of the present invention to provide the program as described above, wherein the sympathetic activator is at least one of capsaicinoid and capsinoid.

It is a further object of the present invention to provide the program as described above, wherein the sympathetic activator is a member selected from the group consisting of a capsicum plant body, a food, a beverage, a pharmaceutical, a cosmetic, a piece of clothing, and a healthcare product wherein contain at least one of capsaicinoid and capsinoid.

It is a further object of the present invention to provide the program as described above, wherein the sympathetic activator is selected from the group consisting of capsiate, dihydrocapsiate, nordihydrocapsiate and a mixture thereof.

It is a further object of the present invention to provide the program as described above, wherein the biological reaction index is either a sympathetic activity reaction index or a parasympathetic activity reaction index.

It is a further object of the present invention to provide the program as described above, wherein the predictive value of the diet effect is selected from the group consisting of the amount of weight loss, the amount of body fat percentage loss, the amount of body fat amount loss, and the amount of total fat area loss.

It is a further object of the present invention to provide the program as described above, wherein the biological reaction index is a sympathetic activity reaction index, the predictive value of the diet effect is an amount of weight loss, and the regression equation is represented by the following (Equation 1):
Y=aX+b (a is between about 0.0026 and about 0.032, and b l is between about −2.9 and about 1.12). (Equation 1)

It is a further object of the present invention to provide a computer-readable recording medium, including the program as described above recorded thereon.

### BRIEF DESCRIPTION OF THE DRAWING

In the accompanying drawing:

FIG. 1 shows a relationship between a sympathetic activity reaction index in a case of ingesting CH19 sweet containing a large amount of capsiate and an amount of weight loss.

### DETAILED DESCRIPTION OF THE PREFERRED EMBODIMENTS

According to principles of the present invention, it is possible to preliminarily determine a diet effect before the initiation of a diet, to propose a diet method more effective for a person who wants to diet, and to preliminarily know a predictive value of the diet effect such as weight loss, so that motivation for diet can be increased.

Hereinafter, embodiments exemplifying principles of the present invention will be described in detail.

### EXEMPLARY METHODS OF THE PRESENT INVENTION

An exemplary method of the present invention provides a method of predicting a diet effect caused by a sympathetic activator in a human.

A first step of a method of the present invention is intended to make a human ingest a sympathetic activator.

Sympathetic Activator

In the context of the present invention, a sympathetic activator encompasses all medicines and foods as long as it is a substance that enhances a sympathetic activity. In particular, a sympathetic activator preferably includes a substance that is expected to have a diet effect. Specific examples include capsaicinoid, capsinoid, synephrine, etc.; preferably, the sympathetic activator is at least one of these substances. More preferably, it is either or both of capsaicinoid and capsinoid. In particular, capsinoid is preferable because it has no pungency and invasive activity.

Examples of capsaicinoid in the present invention include capsaicinoid compounds, which are vanillyl alcohol fatty acid amides such as capsaicin, dihydrocapsaicin, and nordihydrocapsaicin. Preferably, the capsaicinoid is capsaicin. Meanwhile, capsinoid in the present invention refers to a vanillyl alcohol fatty acid ester including, but not limited to, capsiate, dihydrocapsiate and nordihydrocapsiate, those confirmed as components of capsicums, and further including vanillyl alcohol straight-chain fatty acid ester such as vanillyl decanoate
[4-hydroxy-3-methoxybenzyl decanoate (caprate)], vanillyl nonanoate
[4-hydroxy-3-methoxybenzyl nonanoate], vanillyl octanoate
[4-hydroxy-3-methoxybenzyl-octanoate]. Of these, preferable compounds include capsiate, dihydrocapsiate, and nordihydrocapsiate, because non-pungent capsicum contains large amounts of the compounds as its components. Those compounds have the following structural formulae.

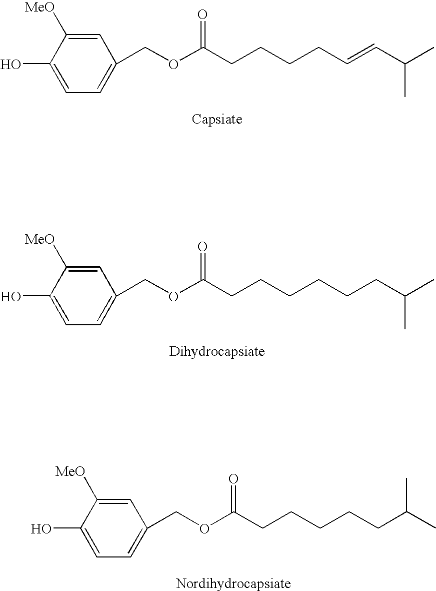

Another aspect of the present invention includes that a sympathetic activator may contain either or both of capsaicinoid and capsinoid as its components. That is, examples of a preferable form include a plant body and/or a fruit of a plant that contains either or both of capsaicinoid and capsinoid and belongs to the genus Capsicum (hereinafter, referred to as “capsicum”), capsicum extract, capsicum product, synthesized capsinoid, etc. Examples of the forms of the capsicum extract and synthesized capsinoid include, but are not limited to, a tablet, capsule, etc., while examples of the capsicum product include, but are not limited to, a capsicum-dried product, capsicum oil, etc. In addition, examples of a preferable form include, but are not limited to, a food, beverage, pharmaceutical, cosmetic, clothing, healthcare product, etc., which contain either or both of capsaicinoid and capsinoid.

A capsicum that contains the above-described capsinoid may be derived from a conventional capsicum variety having pungency such as “Nikko” or “Goshiki”, but any species of capsicum may be used as long as the capsicum contains capsinoid. Particularly, a conventional non-pungent variety of capsicum such as “CH-19 sweet”, “Manganji”, “Fushimiamanaga”, shishitou, or green pepper contains a large amount of capsinoid (capsiate, etc.), so that it may be suitably used. Moreover, “CH-19 sweet”, which is a non-pungent variety, is particularly preferably used because its capsinoid content is high. Herein, the term “CH-19 sweet” includes “CH-19 sweet” and progenies of relative varieties derived from “CH-19 sweet”, etc., and in the present specification, the term “CH-19 sweet” includes all of them.

Capsinoid in the present invention may be obtained from a capsicum plant body containing capsinoid as described above. That is, capsinoid may be separated and purified as follows: ethanol extraction of a capsicum plant body is performed under acidic conditions, and the extracted product is adsorbed to an adsorbent and eluted with ethanol containing an acidic substance, followed by vacuum concentration thereby separating and isolating capsinoid (JP 2004-18428 A). In addition, capsinoid may be synthesized by, for example, a transesterification reaction using a corresponding fatty acid ester and vanillyl alcohol as starting materials, as described in JP 11-246478 A. Alternatively, capsinoid may be synthesized based on its structural formula by other reaction techniques that are known by a person skilled in the art. Moreover, capsinoid may be easily prepared by a synthetic method using an enzyme. That is, for example, in accordance with the method described in JP 2000-312598 A, a desired capsinoid compound may be obtained by using a reverse reaction of lipase using a fatty acid ester corresponding to the desired compound and/or a compound having such fatty acid such as triglyceride and vanillyl alcohol as substrates. Meanwhile, the quantitative analysis of the capsinoid may appropriately be performed by high-performance liquid chromatography (HPLC). Conditions of HPLC may easily be set as long as they are set by a person skilled in the art, but it may be performed by a method described in, for example, JP 2004-18428 A.

Alternatively, capsaicinoid in the present invention may also be obtained from a capsicum plant body containing capsaicinoid, while it may be obtained by a method descried in “Capsicum —Science of Pungency—”, edited by Kazuo Iwai/Tatsuo Watanabe, p. 49-52. Note, also, that capsaicin is commercially available from Sigma-Aldrich Co.

Examples of the ingestion in the present invention include internal use to orally take the substance in a body using a tablet, capsule, etc. and external use to take a substance in a body by (percutaneously) putting or applying the substance with a lotion, ointment, etc. That is, the ingestion in the present invention includes percutaneous ingestion using a blended fiber, etc. prepared by blending the above-described sympathetic activator in a fiber used for clothing, healthcare product, or the like.

A second step of the method of the present invention is intended to obtain a biological reaction index from a human who has ingested a sympathetic activator.

Biological Reaction

The “biological reaction” as used herein means a protective reaction to maintain homeostasis of a living body for invasion. Specific examples thereof include, but are not limited to, autonomic activity reactions such as a sympathetic activity reaction and a parasympathetic activity reaction. In the present invention, the biological reaction may include reactions which have occurred in a human by ingesting a sympathetic activator, such as changes in a skin temperature, pupil, cardiac rate, and respiratory quotient, etc.

Biological Reaction Index

In the present invention, the biological reaction index is represented as a ratio between various responses in a living body in a case of ingesting the above-described sympathetic activator and those in a usual state. That is, an autonomic activity index (sympathetic activity reaction index or parasympathetic activity index) means a ratio between various sympathetic (parasympathetic) activity reactions in a case of ingesting the above-described sympathetic activator and those in a usual state. Specific examples of a method of measuring those sympathetic (parasympathetic) activity reactions include, but are not particularly limited to, pupil photograph by infrared ray, thermal sweating test, measurement of skin temperature, Schirmer test, chewing gum test, pilocarpine test, electrocardiogram, discipline test, heart rate variability power spectral analysis method, etc. Preferable is the heart rate variability power spectral analysis method.

Hereinafter, there will be described in detail a method of measuring an autonomic activity (sympathetic activity and parasympathetic activity) using the heart rate variability power spectral analysis method. First, an electrocardiogram is recorded using a program written by HTBasic (created by professor Toshio Moriya, Course of Human and Environmental Studies, Faculty of Integrated Human Studies, Kyoto University) via an amplifying device (biologically amplified signal BBA-42: manufactured by Biotic Ltd.) and an A/D converter (A/D converter PS-2032GP: manufactured by TEAC Corporation) through electrodes (disposable electrode P-00-S-B: manufactured by GE Marquette Medical Systems Japan) connected to a subject. The R-R intervals obtained from the electrocardiogram are sampled at 2 Hz, and the DC component and trend are eliminated from 256 seconds of data using a numerical filter, followed by passing through a Hamming-type data window to perform fast Fourier transform. Thereafter, in accordance with the method by Moriya et al. (Tatsuya Hayashi, Izuru Masuda, Minoru Shinohara, Toshio Moriya, Kazuwa Nakao: Analysis of autonomic dynamics by power spectral analysis of heart rate variability, exercise biochemistry, 1994 Vol. 6: 30-37), a frequency domain ranging from 0.03 to 0.15 Hz and a frequency domain ranging from 0.15 to 0.8 Hz are defined as a Low component and a High component, respectively, and states of sympathetic and parasympathetic activities are quantified from integrated values of respective spectra and a Total (total autonomic activity index) integrated value of a spectrum obtained by adding the Low component and High component. That is, the High/Total ratio and the Low/High ratio are represented as a parasympathetic activity index (PNS index) and a sympathetic activity index (SNS index), respectively. Therefore, the above-described autonomic activity reaction index (sympathetic activity reaction index or parasympathetic activity reaction index) may be calculated as “quantified sympathetic (parasympathetic) activity index in a case of ingesting a sympathetic activator”/“quantified sympathetic (parasympathetic) activity index in a usual state” using such quantified parasympathetic activity index or sympathetic activity index.

Meanwhile, the respiratory quotient (RQ) index may also be represented as a ratio between a respiratory quotient in a case of ingesting the above-described sympathetic activator and that in a usual state. Herein, the respiratory quotient is represented as a ratio between the amount of carbon dioxide released and the amount of oxygen absorbed from the outside world when oxygen respiration occurs in a living body, that is, represented as [CO2]/[O2] ([] is a number of moles of the substance).

When glucose (sugar) is completely burned as a respiratory substrate, the RQ value is experimentally approximately 1. In the case of a substance such as a protein or fat, which is a molecule in which the ratio of bound oxygen is less than that in a sugar, RQ is less than 1. Meanwhile, when malic acid or oxalic acid serves as a respiratory substrate, RQ is larger than 1. That is, measurement of the value enables estimation of a ratio of used lipids/carbohydrates in a living body. Note that the amounts of carbon dioxide and oxygen may be easily measured using a known instrument (carbon dioxide concentration meter, oxygen concentration meter).

Of those indices, a sympathetic activity reaction index is preferably used.

A third step of the method of the present invention is to calculate a predictive value of a diet effect from the aforementioned biological reaction index obtained in the second step, based on a regression equation representing the relationship between a preliminarily determined biological reaction index and diet effectiveness.

Diet Effectiveness

In the present invention, the diet effectiveness may be any change produced in a living body by making a human ingest a sympathetic activator. From a quantitative viewpoint, examples thereof include the amount of weight loss, amount of body fat percentage loss, amount of body fat amount loss, and amount of total fat area loss. Preferable is the amount of weight loss. The diet effectiveness is preferably represented as a ratio between amounts before and after ingesting a sympathetic activator. For example, when measuring the amount of weight lossit is represented as (weight before ingesting a sympathetic activator—weight after ingesting a sympathetic activator)/(weight before ingesting a sympathetic activator).

In this case, the effective amount after a lapse of a predetermined diet period is predicted in the case that a regression equation based on data measured after a lapse of such period is used, and the period to obtain the data for formulating the regression equation may be about 1 week to about 6 months, preferably about 2 weeks to about 3 months. Note that determination of the effectiveness using a regression equation may be performed by at least single ingestion of a sympathetic activator.

The diet effectiveness may be measured by a known method, but weight and body fat percentage are preferably measured using precision scales (10 g unit) and BODPOD (trademark: manufactured by Life Measurement, Inc.), respectively. Meanwhile, a total fat area is preferably measured using a CT scan.

Predictive Value of Diet Effect

The predictive value of a diet effect of the present invention can be calculated by applying the biological reaction index obtained in the second step to a regression equation which represents a correlation between a preliminarily determined biological reaction index and diet effectiveness. The regression equation representing a correlation between a preliminarily determined biological reaction index and diet effectiveness can be obtained from the above-described biological reaction indices and diet effectiveness in a case of ingesting a sympathetic activator, and which have been obtained for a certain number of subjects, by general statistics and analysis techniques, such as the method of least squares. Note that the fact that there is a correlation between the biological reaction index and the diet effectiveness indicates that a determination coefficient is significantly large, and the significantly large determination coefficient generally means that the determination coefficient R2 is 0.6 or more, preferably 0.65 or more.

Therefore, a predictive value of a diet effect may be obtained by substituting a biological reaction index obtained from a human who has ingested a sympathetic activator for the above-described regression equation.

These mathematical processes can be performed by using a commercially available calculation software such as EXCEL (product name, Microsoft Corporation).

### PROGRAM OF THE PRESENT INVENTION

The present invention also provides a program for performing the method of predicting a diet effect of the present invention. The program of the present invention is a program for predicting a diet effect caused by a sympathetic activator and making a computer perform a method of predicting a diet effect, which involves calculating a predictive value of the diet effect from a biological reaction index obtained by a human who has ingested the sympathetic activator based on a regression equation showing a correlation between the preliminarily determined biological reaction index and diet effectiveness. In the context of the present invention, the program includes a set of instructions, e.g., logic, executable by a general purpose computing device.

Another embodiment of the present invention relates to a computer-readable recording medium, e.g., a memory, in or on which the above-described program has been recorded.

The procedure of the present invention is a procedure for performing the third step of the method of predicting a diet effect of the present invention. A program executable by a computer to perform such a procedure may be created in accordance with usual programming or scripting techniques.

A program according to the present invention may be stored in a computer-readable recording medium. Herein, the “recording medium” includes, but is not limited to: any “portable physical medium” such as a FLOPPY (trademark) disk, magnetic optical disk, ROM, CD-ROM, MO, or DVD; any “fixed physical medium” such as ROM, RAM, or HDD that are integrated in various computer systems; or a “communication medium” to maintain a program for a short time such as a communication line or carrier wave in the case that a program is sent through a network including LAN, WAN, or internet.

The “program” includes a data processing method written in any language or writing method, and is not limited to the format of source code, binary code, etc. The “program” is not necessarily limited to a program constructed as a single form and includes a program dispersively constructed as plural modules or libraries and a program to accomplis its functions in cooperation with another program, including, but not limited to, an OS (operating system). For any specific construction, reading procedure, or install procedure after reading a recording medium, in each apparatus shown in the embodiments, known constructions or procedures may be used.

### EXAMPLES

Hereinafter, the present invention will be described in more detail by way of the following non-limiting examples, but the technical scope of the present invention is not limited to the present examples.

### Example 1

Relationship between Sympathetic Activity Reaction Index and amount of weight loss in a case of ingesting “CH-19 sweet” containing a large amount of capsiate

12 healthy male subjects were divided into two groups of a “CH-19 sweet” group (n=7, age 32.29±11.84) and a control group (n=5, age 27.80±7.66). Experiments were performed in a meal amount control period (for 9 days) and a “CH-19 sweet” ingestion period (for 14 days). The subjects of the control group ingested no “CH-19 sweet”. During the experimental period, the subjects ate the same three meals every day (Table 1). In addition, they were banned from drinking alcohol, exercising, and eating meals other than the provided meals, but they were allowed to drink water freely. Note that the meal menu was changed depending on preference of each subject, and the subjects were permitted to ingest a certain amount of favorite food determined by the subjects themselves every day. Meal amounts at the start of the experiment were determined by subtracting the energy amounts of their favorite foods from the respective energy requirements (example: Physiological Intensity of Life Activity II, age 18 to 29, average weight 64.7 kg, 2,300 kcal) calculated from “Japanese nutritional requirement (the 6th revision)” determined by Ministry of Health, Labor and Welfare. By using the subtracted energy amounts as bases, the meal amounts were determined in 9 days by increasing or decreasing the amounts of rice and bread. As a result, the numbers of calories including those from the favorite foods were 2,372±419 kcal (the “CH-19 sweet” group) and 2,454±239 kcal (the control group), and there were no significant differences (P=0.707). For the “CH-19 sweet” group, the subjects ingested “CH-19 sweet” (0.4/kg·wt/day) three times on the basis of the average of the weights for the last three days of the meal amount control period. “CH-19 sweet” was ingested in a frozen state (the capsiate content in “CH-19 sweet”: average 2,348 μg/gDW). For the control group, the subjects ingested no additional food and continuously ate the same meals. The amount (rate) of weight loss was calculated as a percentage (%) of (B-A)/B (Equation 2), wherein A is a weight measured after completion of the “CH-19 sweet” ingestion period (for 14 days), and B is an arithmetic average weight for the last three days of the meal amount control period. Note that the weights were measured by a digital scale product name: Digital Platform Scale FW100KAI, manufactured by A & D Co., Ltd.).

On the other hand, autonomic activities were measured a total of two times (once during the meal amount control period, once during the “CH-19 sweet” ingestion period) during lunch. For 7 subjects of the “CH-19 sweet” group, after the autonomic activities in the rest state were measured for about 15 minutes, the subjects ingested only meals for lunch during the meal amount control period, and the autonomic activities were measured for about 15 minutes. Meanwhile, during the “CH-19” sweet ingestion period, the autonomic activities in the rest state were measured for about 15 minutes, and the subjects ingested “CH-19 sweet” and meals for lunch, and the autonomic activities were measured for about 60 minutes. The autonomic activities were measured using the above-described heart rate variability power spectral analysis method. From those measurement results, the sympathetic activity reaction index in a case of ingesting “CH-19 sweet” was calculated as a percentage (%) obtained by dividing the sympathetic activity reaction index after ingesting “CH-19 sweet” and food for lunch by the sympathetic activity reaction index in a usual state (after ingesting only meals for lunch during the meal amount control period).

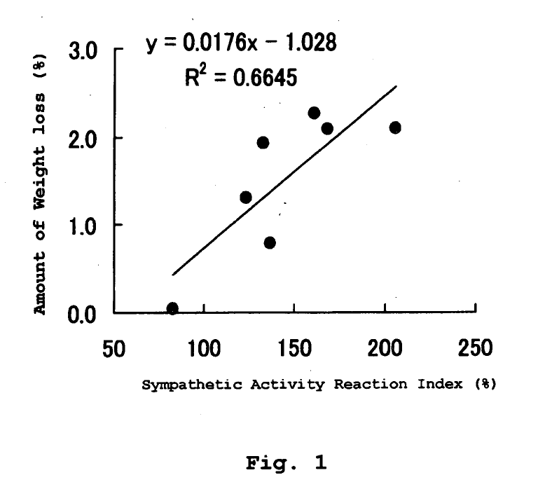

From the results of the experiment, when ingesting “CH-19 sweet”, there was a tendency for the Low/High ratio which is a sympathetic activity reaction index to increase, while there was a tendency for the High/Total ratio which is a parasympathetic activity reaction index to decrease. The results revealed that the sympathetic activity was enhanced and the parasympathetic activity was suppressed when ingesting “CH-19 sweet”. Meanwhile, the sympathetic activity reaction indices of the subjects who had no weight loss tended to differ from those of other subjects. The results revealed that such subjects had low sensitivities to “CH-19 sweet”. In addition, for 7 subjects of the “CH-19 sweet” group, there was a positive correlation between the amount of weight loss and the sympathetic activity reaction index when ingesting “CH-19 sweet”, and the regression equation (Equation 3) (Y=0.0176x−1.028, determination coefficient (R2) is 0.6645) was obtained (FIG. 1). In addition, from the 95% confidence interval of the regression line, the above-described equation can be represented as (Equation 4) Y=ax+b (a is a number of 0.0026 or more and less than 0.032, b is a number from −2.9 to 1.12).

The sympathetic activity reaction indices obtained from the subjects who had ingested “CH-19 sweet” were substituted into (Equation 3) above, and the resultant Y value was determined as a predictive value of the amount of weight loss (%). This enabled preliminary prediction of a diet effect before the initiation of a diet.

TABLE 1

| | |
|---|---|
| Breakfast | Bread roll |
| | Graining corn cream soup |
| | A pack of loin rolls (50 g) |
| | Tomato (50 g) |
| | Cucumber (100 g) |
| | A bag of mayonnaise (12 g) |
| | A cup of yogurt |
| Lunch | Rice |
| | A pack of ingredients of Chuka-don (product name) |
| | Milk (200 ml) |
| | Cabbage (75 g) |
| | A bag of dressing (15 g) |
| Dinner | Rice |
| | Seaweed miso soup |
| | A coarse grind of hamburger |
| | A tofu-hijiki hamburger |
| | A beef-lotus root cutlet |
| | A fried chicken |
| | Two ankake-meat balls |
| | Two jiao-zi |
| | A cooked food of chicken white meat and hijiki |
| | A chicken-kinpira-gobou |
| | A steamed egg custard |
| | Tomato (50 g) |
| | Cabbage (75 g) |
| | A bag of dressing (15 g) |

Tea (500 ml) per day

While the invention has been described in detail with reference to the preferred embodiments thereof, it will be apparent to one skilled in the art that various changes can be made, and equivalents employed, without departing from the scope of the invention. Each of the aforementioned documents, including the foreign priority document JP2004-258935, filed Sep. 6, 2004, are hereby incorporated by reference.

What is claimed is:

1. A method of predicting diet effect caused by a sympathetic activator comprising:
a) making a human ingest the sympathetic activator;
b) obtaining a biological reaction index from said human; and
c) calculating a predictive value of the diet effect from the biological reaction index, based on a regression equation which shows a correlation between the preliminarily determined biological reaction index and diet effectiveness.

2. The method according to claim 1, wherein the sympathetic activator is at least one of capsaicinoid and capsinoid.

3. The method according to claim 1, wherein the sympathetic activator is selected from the group consisting of a capsicum plant body, a food, a beverage, a pharmaceutical, a cosmetic, a piece of clothing, and a healthcare product, wherein said activator contains at least one of capsaicinoid and capsinoid.

4. The method according to claim 1, wherein the sympathetic activator is selected from the group consisting of capsiate, dihydrocapsiate, nordihydrocapsiate and a mixture thereof.

5. The method according to claim 1, wherein the biological reaction index is either a sympathetic activity reaction index or a parasympathetic activity reaction index.

6. The method according to claim 1, wherein said predictive value of the diet effect is selected from the group consisting of the amount of weight loss, the amount of body fat percentage loss, the amount of body fat amount loss, and the amount of total fat area loss.

7. The method according to claim 1, wherein the biological reaction index is a sympathetic activity reaction index, the predictive value of the diet effect is an amount of weight loss, and the regression equation is represented by the following (Equation 1):
Y=ax+b (Equation 1)
wherein a is between about 0.0026 and about 0.032; and
wherein b is between about −2.9 and about 1.12.

8. A system for predicting a diet effect caused by a sympathetic activator and executable by a computing device to predict a diet effect caused by a sympathetic activator, the system comprising:
means for calculating a predictive value of the diet effect from a biological reaction index obtained by a human who has ingested the sympathetic activator, based on a regression equation showing a correlation between the preliminarily determined biological reaction index and a diet effectiveness.

9. The system according to claim 8, wherein the sympathetic activator is at least one of capsaicinoid and capsinoid.

10. The system according to claim 8, wherein the sympathetic activator is selected from the group consisting of a capsicum plant body, a food, a beverage, a pharmaceutical, a cosmetic, a piece of clothing, and a healthcare product, wherein said activator contains at least one of capsaicinoid and capsinoid.

11. The system according to claim 8, wherein the sympathetic activator is selected from the group consisting of capsiate, dihydrocapsiate, nordihydrocapsiate and a mixture thereof.

12. The system according to claim 8, wherein the biological reaction index is either a sympathetic activity reaction index or a parasympathetic activity reaction index.

13. The system according to claim 8, wherein the predictive value of the diet effect is selected from the group consisting of an amount of weight loss, amount of body fat percentage loss, amount of body fat amount loss, and amount of total fat area loss.

14. The system according to claim 8, wherein the biological reaction index is a sympathetic activity reaction index, the predictive value of the diet effect is an amount of weight loss, and the regression equation is represented by the following (Equation 1):
Y=aX+b (Equation 1)
wherein a is between about 0.0026 and about 0.032; and
wherein b is between about −2.9 and about 1.12.

15. A computer-readable recording medium, comprising the system according to claim 8 recorded thereon.
